(19) United States
(12) Patent Application Publication
(10) Pub. No.: US 2024/0207157 A1
(43) Pub. Date: Jun. 27, 2024

(54) ORAL CARE AGENT

(71) Applicant: DR. KURT WOLFF GMBH & CO. KG, Bielefeld (DE)

(72) Inventors: Erik SCHULZE ZUR WIESCHE, Bielefeld (DE); Joachim ENAX, Bielefeld (DE); Frederic MEYER, Bielefeld (DE)

(73) Assignee: DR. KURT WOLFF GMBH & CO. KG, Bielefeld (DE)

(21) Appl. No.: 18/288,082

(22) PCT Filed: Apr. 28, 2022
(86) PCT No.: PCT/DE2022/100317

§ 371 Date: Oct. 24, 2023

(30) Foreign Application Priority Data
May 3, 2021 (DE) 10 2021 111 354.7

(51) Int. Cl. A61K 8/49; A61K 8/24; A61Q 11/00

(52) U.S. Cl. CPC A61K 8/4953; A61K 8/24; A61K 8/4946; A61Q 11/00; A61K 2800/30

(57) ABSTRACT

An oral care agent, in particular toothpaste or mouthwash, contains a methylxanthine, selected from the group consisting of caffeine, theobromine and theophylline, and a calcium phosphate compound, preferably in particulate form, selected from the group consisting of monocalcium phosphate monohydrate (MCPM), anhydrous monocalcium phosphate (AMCP), octacalcium phosphate (OCP), amorphous calcium phosphate (ACP), calcium-deficient hydroxyapatite (CDHA), hydroxyapatite (HA or HAP) and tetracalcium phosphate (TTCP), particularly preferably hydroxyapatite. The addition of methylxanthines, of which preferably caffeine is used, in combination with the other compounds achieves a synergistic effect.

### CROSS REFERENCE TO RELATED APPLICATIONS

This application is the National Stage of PCT/DE2022/100317 filed on Apr. 28, 2022, which claims priority under 35 U.S.C. § 119 of German Application No. 10 2021 111 354.7 filed on May 3, 2021, the disclosure of which is incorporated by reference. The international application under PCT article 21(2) was not published in English.

### BACKGROUND OF THE INVENTION

### 1. Field of the Invention

The present invention relates to an oral care agent, in particular toothpaste or mouthwash.

### 2. Description of the Related Art

In combination with a toothbrush, a toothpaste can be used to mechanically clean teeth and gums, and is a soft or semi-solid composition for oral use. An oral gel is applied in the same way, but has a gel-like composition.

A mouthwash, also called a mouth rinse, is a liquid formulation that can be used, among other things, for preventing caries and other diseases in the oral cavity.

Simply because of the increasingly high-carbohydrate diet worldwide, dental care is becoming more and more important. Therefore, in addition to aesthetic aspects, particular emphasis is increasingly being placed on preventive care, wherein the focus is primarily on reducing or even avoiding plaque, caries, and/or halitosis (bad breath) as well as healthy gums.

One of the features of the gums is that they surround the teeth cervically. This means that the gums surround the neck of the tooth, sealing the entry point of the tooth into the jawbone of the oral cavity. The gums therefore serve, among other things, to protect the tooth and hold it in place.

The different parts of a natural tooth are the crown, neck and root, wherein these are made up of several layers. Of these layers, you normally only see the outer enamel (enamelum), which surrounds the dentin and other layers. For example, in order to be able to bite or grind food without damaging the teeth, the enamel is very hard. It consists of about 97% by weight of hydroxyapatite (HAP), which has the molecular formula Cas(PO₄)₃(OH). The dentin is also considered to be hard tooth tissue and also consists of about two-thirds hydroxyapatite. In addition to hydroxyapatite, dentin also contains proteins (e.g. collagen) and water and is therefore softer than enamel.

Dental diseases such as caries can be based on the formation of bacterial microfilms and/or be due to bacterial inflammation. Although often preventable through screening, tooth decay remains one of the most common chronic diseases in children and adults, and there is a great need for products to prevent and cure tooth decay worldwide.

It has been documented that saliva has a remineralizing effect due to its supersaturation of Ca²⁺- and PO₄³⁻ ions in bioavailable form. However, the remineralizing effect caused by saliva is not only slow, but also apparently insufficient to protect people from caries and/or to remineralize already existing tooth lesions without the addition of mineralization-enhancing additives.

Caries is considered to be a disease dependent on several influences, in particular tooth enamel and dentin, with the involvement of microorganisms. Thus, in a first step, a precipitate, also called pellicle, can form on the tooth surface from salivary protein, among other things. The rougher the tooth surface, the easier it is for the deposit to form or adhere thereto. After some time, a film a few micrometers thick covering the tooth surface is formed from this pellicle. Bacteria can then continue to multiply and spread on this film, wherein this film can be considered a biofilm. Furthermore, the bacteria mentioned can produce organic acids, such as gluconic and lactic acid, from low-molecular carbohydrates that are frequently found in today's food. Both these organic acids produced by bacteria and those supplied through food, such as fruit acids, can cause erosion of the tooth structure. Among other things, calcium phosphates are dissolved from the enamel. If this process is not stopped or, preferably, even reversed, demineralization of the enamel and possibly also of the dentin can occur after some time. A defect in the tooth structure that has developed in this way is called a carious lesion, wherein, for example, carious lesions on the dentin layer can be recognized by a yellow to brown discoloration of the corresponding part of the tooth. So, while carious lesions on the enamel alone can often still be reduced or eliminated by remineralizing the enamel, in the case of carious lesions that affect other layers besides the enamel, the damaged part of the tooth is usually removed and the resulting hole (caries lesion) is sealed with a filling material. In summary, it can be said that caries formation can be promoted by the interaction of the following factors:
particular bacteria adhering to the pellicle, low tooth quality or mineral quality of the tooth, plaque
food containing low molecular weight carbohydrates
time.

Accordingly, one possibility for preventing caries could be the supply of calcium phosphate compounds, in particular hydroxyapatite. In addition, there are various approaches, all aimed at preventing caries in order to avoid the aforementioned treatments. Preventing caries with fluoride-containing dental care products can be regarded as the current standard. J. M. ten Cate: “Contemporary perspective on the use of fluoride products in caries prevention”, British Dental Journal, 23 Feb. 2013, vol. 214, no. 4, p. 161-167, describes the use and mode of action of fluoride-containing dental care products such as toothpaste and mouthwash in a review paper. It is particularly noted that with these fluoride-containing dental care products, the reduction of caries is achieved by their regular use. As a model, it is assumed here that fluoride accelerates the natural mineralization from saliva.

Various fluoride compounds, such as sodium fluoride, stannous fluoride, amine fluorides and sodium monofluorophosphate) are suitable as fluoride sources in the dental care products mentioned. J. M. ten Cate: “The Need for Antibacterial Approaches to Improve Caries Control”, Adv Dent Res 21:8-12, August 2009, p. 8-12, deals with the fact that fluoride supply alone may not be sufficient to achieve adequate caries prevention because, as mentioned above, bacteria also play a crucial role in caries formation. However, the fluoride ion itself does not show a pronounced antimicrobial effect against bacteria that can cause caries. For this reason, compositions for preventing caries are being considered that, in addition to fluoride, should also contain one or more antimicrobial substance. One example is chlorhexidine (CHX), the antimicrobial effect of which in the oral cavity has been investigated in many studies.

However, the use of fluorides in dental care products is also controversial, as negative side effects are feared. One of these side effects is dental fluorosis, which is caused by excessive fluoride intake during tooth formation. Acute toxic effects include nausea, vomiting and diarrhea. Other examples are bone fluorosis, which is manifested by thickening of the outer bone layer and the associated loss of elasticity and resilience of the bones, and enamel fluorosis, which is recognizable by the appearance of whitish enamel spots on the tooth surface. In addition, it has been reported that swallowing high-dose dental care products can cause acute fluoride poisoning, especially in children, which can occasionally even be fatal (Bashash, M. et al. Prenatal fluoride exposure and cognitive outcomes in children at 4 and 6-12 years of age in Mexico. Environ. Health Perspect. 125, 097017 (2017)). Furthermore, it is reported that the WHO is not able to set a value for a daily fluoride requirement because fluoride is not an essential trace element and thus there are no diagnostic parameters and no evidence for the existence of clinical symptoms of “fluoride deficiency”.

The use of an antimicrobial substance such as chlorhexidine is also controversial in dental care, in particular in preventing caries. It has been reported that a relevant effect for preventing caries is not always reproducible and occurs only in a part of the cases investigated. Furthermore, the antimicrobial effect of chlorhexidine is not limited to the oral bacteria involved in caries formation, but also includes beneficial bacteria. In addition, long-term treatment with chlorhexidine-containing products leads to undesirable side effects, such as tooth discoloration and taste disorders.

Biomimetic tooth and mouth rinses with artificial enamel may contain e.g. zinc carbonate hydroxyapatite. This zinc carbonate hydroxyapatite is also commercially known as microrepair. Zinc carbonate hydroxyapatite-based products can reduce initial bacterial colonization on the enamel surface without having antimicrobial properties that may disturb the ecological balance of the oral cavity. Moreover, these products are supposed be used, among other things, for remineralization and the repair of microfine defects in the tooth enamel as well as the formation of a protective layer.

Due to the above limitations of both the homeostatic mechanism through saliva and fluoride addition-based approaches to caries prevention and mineralization, there is a need for alternative strategies that are at least equivalent to the efficiency of fluoride addition in mineralization, but without the corresponding undesirable side effects.

Furthermore, there are biometric dental care products based on the use of hydroxyapatite. For example, DE 10 2020 001 823 A1, WO 2018/024649 A1, DE 10 2017 009 626 A1 and DE 10 2018 102 365 describe oral care compositions containing synthetic hydroxyapatite, wherein hydroxyapatite, as indicated above, is a bioactive and biocompatible material with a similar chemical composition to the apatite of human tooth enamel.

Methylxanthines, especially caffeine, theobromine and theophylline, are also used in many areas of cosmetics and personal hygiene due to their stimulating effect.

DE 298 06 937 U1 describes a toothpaste to which one or more stimulating substances are added, which may be, for example, caffeine or nicotine.

DE 201 19 966 U1 also describes a toothpaste enriched with invigorating substances, wherein caffeine or other stimulating substances are included in the toothpaste formulation.

A caffeine-containing toothpaste is also known from JP 2010/275261 A1.

The publication “Neurophysiological effect of flavor and caffeine added to toothpaste”, Sangyo Eiseigaku Zasshi, vol. 52, issue 4, pages 172-181, 2010:1478424 CAN156:675063 also discloses enriching toothpaste with caffeine, among other things, to reduce fatigue.

WO 2021/013283 A1 discloses compositions for oral hygiene means, in particular toothpastes, which contain caffeine and lysine-containing bitter blockers. Since oral hygiene products are usually not swallowed but spat out, the caffeine must be present in a sufficiently high concentration for it to be effective. The bitter blockers serve to achieve a pleasant taste experience, even at these very high caffeine concentrations.

Finally, a mouthwash containing caffeine is also offered by Johnson & Johnson under the name “LISTERINE GREEN TEA”.

However, the dwell time of toothpaste or mouth rinse/mouthwash in the oral cavity is limited to about 30 seconds to a maximum of 3 minutes, so that only little caffeine can interact with the gums during the duration of dental care. Afterwards, these are usually spat out and also rinsed with water. This causes the effective concentration of caffeine in the oral cavity to decrease very quickly. Such oral hygiene products serve to care for, moisten and kill bacteria, but have no long-term positive effect on the cells of the gums.

Caffeine has a positive influence on the cardiovascular system. In the Spyridopoulos et al. (2008) study, Arterioscler Thromb Vasc Biol, 28, 1967-1974, it was shown that in humans, after drinking four cups of coffee, a caffeine concentration of 20-50 micromol/liter is reached in the blood serum, which corresponds to a total amount of 15-35 milligrams.

Caffeine in these physiological concentrations led to functional improvements in the cells of the innermost layer of the blood vessel wall, the endothelial cells, and their precursors. Interestingly, these phenomena were dependent on intact mitochondria, which assigns a central role to these cell organelles in the functional improvement of endothelial cells by caffeine.

Mitochondria provide the energy needed for the body in the form of the molecule adenosine triphosphate (ATP). The substrates required for this, so-called reduction equivalents (NADH⁺H⁺ and FADH₂), come from the citrate cycle and fatty acid oxidation—processes that also take place in the mitochondria.

The reduction equivalents are introduced into the respiratory chain, where they are used to reduce oxygen to water. As a by-product of the respiratory chain, oxygen radicals are continuously produced that can damage macromolecules such as proteins, lipids and the DNA present in the mitochondria and ultimately damage the functioning of the mitochondria.

The production of oxygen radicals increases with age and is also increased in cardiovascular diseases and type 2 diabetes mellitus, which explains the loss of mitochondrial function both in old age and in the diseases mentioned.

Recent studies provide clues as to the molecular mechanism of caffeine on mitochondria. According to recent studies, caffeine has an enhancing effect on the import of p27 (CDKNIB, “Cyclin Dependent Kinase Inhibitor IB”) into mitochondria. This was accompanied by an improvement in the function of various cell types in the heart. In this way, heart muscle cells were protected from cell death, as occurs in heart attacks. Furthermore, connective tissue cells in the heart, cardiac fibroblasts, could better differentiate into myofibroblasts. Myofibroblasts compensate for dead heart muscle cells by forming scar tissue. Furthermore, endothelial cells showed an increased ability to migrate, which is essential for the healing of vascular damage. In mice, the administration of caffeine in dependence on p27 was able to restore the functionality of mitochondria from old animals to the level of younger animals. Ultimately, the administration of caffeine in the drinking water of mice reduced damage after an experimental heart attack, which had previously been brought up to the physiological level of a type 2 diabetic by means of special diets. These studies support the findings from epidemiological studies that moderate caffeine consumption has a positive effect on the cardiovascular system.

It has also been known for some time that caffeine increases the analgesic potency of acetylsalicylic acid or paracetamol.

Other uses of caffeine, besides increasing alertness or eliminating fatigue, include circulatory and respiratory stimulation, prevention of bronchopulmonary dysplasia, and other effects on skin and hair.

Oral care agents containing caffeine are also known, wherein these oral care agents are based on the above effects of caffeine.

DE 298 06 937 U1 describes a toothpaste to which one or more stimulating substances are added, which may be, for example, caffeine or nicotine.

DE 201 19 966 U1 also describes a toothpaste enriched with invigorating substances, wherein caffeine or other stimulating substances are included in the toothpaste formulation.

A caffeine-containing toothpaste is also known from JP 2010/275261 A1.

The publication “Neurophysiological effect of flavor and caffeine added to toothpaste”, Sangyo Eiseigaku Zasshi, vol. 52, issue 4, pages 172-181, 2010:1478424 CAN156:675063 also discloses enriching toothpaste with caffeine, among other things, to reduce fatigue.

WO 2021/013283 A1 discloses compositions for oral hygiene means, in particular toothpastes, which contain caffeine and lysine-containing bitter blockers. Since oral hygiene products are usually not swallowed but spat out and rinsed with water, the caffeine can be present in a sufficiently high concentration for it to be effective. The bitter blockers serve to achieve a pleasant taste experience, even at high caffeine concentrations.

A mouthwash containing green tea extract is offered by Johnson & Johnson under the name “LISTERINE GREEN TEA”.

The manufacturer Splat Global also offers a toothpaste called “Energy Mint Whitening Coffee Out Toothpaste”, which contains caffeine, tricalcium phosphate and calcium pyrophosphate as cleaning agents.

The manufacturer Carbon & Clay offers a toothpaste called “Magical Mint CBD Hemp Oil+Silver Toothpaste” that contains theobromine, among other things.

The manufacturer Dr. Kaschny HealthCare offers a mouthwash under the name “Mouthwash with Caffeine” that contains caffeine, among other things.

JP 2010-275 261 A discloses a toothpaste containing xanthine derivatives selected from the group consisting of caffeine, theophylline, theobromine and salts thereof, water, glycerol and a binder. This toothpaste may also contain cleaning agents.

WO 2011/100671 A2 discloses a method and theobromine-containing compositions for increasing the mechanical strength of teeth.

JP 2020037297 A describes compositions for the oral cavity containing xanthine derivatives selected from caffeine, theophylline and theobromine.

From the GNPD [Online] MINTEL database; 19 Sep. 2019, (2019-09-19), anonymous: “Energy Mint Whitening Coffee Out Toothpaste”, XP055947792, Database accession no. 6878779 a toothpaste is known that contains caffeine and calcium pyrophosphate.

U.S. Pat. No. 5,075,115 A describes tablets having 60% theophylline and dicalcium phosphate.

WO 03/063834 A1 describes an oral active substance delivery system, for example in the form of tablets containing theophylline and dibasic calcium phosphate dihydrate. However, there is still a need for an oral care agent to treat or prevent various diseases affecting the teeth.

Toothpaste according to the prior art contains abrasives, also called cleaning agents, which can remove plaque and harmful bacteria from the tooth surface during the tooth cleaning process, usually together with the toothbrush, and also provide a whitening effect. Abrasives or cleaning agents are contained in toothpastes in an amount of up to 15% by weight or more in relation to the total weight of the toothpaste. Examples of abrasives are whiting chalk, marble powder and/or silicate compounds such as silica. The most commonly used worldwide are poly silicas (hydrated silica/silica) and calcium carbonate. The disadvantage is that with the exception of calcium phosphate compounds (e.g. hydroxyapatite) all types of cleaning agents are foreign to the body and do not correspond to the natural tooth mineral (hydroxyapatite). For example, cleaning agents with a high relative hardness can irreparably damage the tooth structure. If the cleaning agent has too low a hardness, the cleaning power of the toothpaste formulation may be too low to effectively remove plaque during tooth brushing (increased risk of caries and periodontitis).

Furthermore, the use of the product should not significantly disturb the ecological balance in the oral region and/or risk tooth discoloration or taste disturbance.

### SUMMARY OF THE INVENTION

The object of the invention is to provide an oral care agent, in particular a toothpaste or a mouthwash, with one or more biomimetic or bioinspired active ingredient, in particular for preventing caries, which avoids the disadvantages of the prior art described above.

In particular, an oral care agent, in particular a toothpaste or mouthwash, is to be provided that mineralizes already existing, minor caries lesions and/or repairs microfine defects in the tooth enamel. Furthermore, a protective layer is to be applied to the tooth and/or open dentinal tubules are to be closed. For this purpose, the above-mentioned effects are to be achieved, wherein the disadvantages that may be associated with the use of fluoride are to be avoided. Furthermore, the adhesion of bacteria to the enamel is to be advantageously reduced without significantly disturbing the ecological balance in the oral cavity and/or risking tooth discoloration or taste disturbance. The oral care agent is also said to have no irritating effect on the mucous membranes.

These objects are achieved with an oral care agent in that the oral care agent contains caffeine and a calcium phosphate compound, preferably in particulate form, selected from the group consisting of monocalcium phosphate monohydrate (MCPM), anhydrous monocalcium phosphate (AMCP), octacalcium phosphate (OCP), amorphous calcium phosphate (ACP), calcium-deficient hydroxyapatite (CDHA), hydroxyapatite (HA or HAP) and tetracalcium phosphate (TTCP), particularly preferably hydroxyapatite.

It has been surprisingly shown in the context of the invention that caffeine in combination with the above-mentioned calcium phosphate compounds, which have a remineralizing effect and are not intended primarily for use as cleaning agents, has a synergistic effect. The addition of caffeine significantly increases the binding of the aforementioned calcium phosphate compounds to the teeth and/or gums.

Conversely, the aforementioned calcium phosphate compounds also bind caffeine to the teeth and/or gums, resulting in a deposit effect. Therefore, in the present case the caffeine is not added to the oral care agent because of its stimulating effect, but because of this synergistic effect, as well as its gum regenerating effect.

The calcium phosphate compound may be present in different crystal forms, including mixtures of these crystal forms, wherein more round crystal forms are preferred over more pointed crystal forms (for example, needle-shaped crystals). Likewise, they can have different crystal and aggregate sizes, also in mixed form, and different crystallinities, also in mixed form.

Other calcium phosphate compounds used as cleaning agents, in particular calcium pyrophosphate, calcium diphosphate and tricalcium phosphate (α-TCP, β-TCP), however, surprisingly proved to be significantly less effective in remineralization, reduction of biofilm formation and reduction of pain-sensitive teeth and gum regeneration in combination with caffeine, such that the present oral care agent is free of the aforementioned compounds. The subjective perception of use with regard to the reduction of pain-sensitive teeth and gum regeneration with caffeine-containing oral care agents with remineralizing calcium phosphate compounds proved to be significantly better than that of caffeine-containing oral care agents with calcium phosphate compounds used as cleaning agents in an application study with 50 participants.

Hydroxylapatite (Cas(PO₄)₃(OH)) is also known as hydroxyapatite. It is a mineral from the mineral class of phosphates, which crystallizes in a hexagonal crystal system. In addition, hydroxyapatite is a member of the apatite group and forms a gapless mixed series with chlorapatite and fluorapatite.

A Cas(PO₄)₃(OH) suitable according to the invention is described, for example, in DE 10 2016 114 189.5. The Cas(PO₄)₃(OH) used according to the invention is preferably produced synthetically. This means that the Cas(PO₄)₃(OH) used according to the invention is preferably not obtained by burning out the organic components from animal material such as bones.

The calcium phosphate compounds used according to the invention can be present both in pure form and in the form of substituted compounds as well as mixtures thereof. According to the invention, a pure form is present if the ions contained in the respective calcium phosphate compound are each substituted by less than 1%, preferably less than 0.5%, even more preferably less than 0.1% by one or more other ions. For example, in pure hydroxyapatite, the Ca²⁺ ions are substituted by, for example, Mg²⁺ or Zn²⁺ to less than 1%, preferably less than 0.5%, even more preferably less than 0.1%.

Further preferably, the calcium phosphate compounds preferably contain no doping, such as zinc carbonate doping. However, doped calcium phosphate compounds can also be used.

The X₅₀ value of the volume-based particle size distribution of the calcium phosphate compounds is 1.0 nm to 100.0 μm, preferably 10 nm to 10.0 μm, more preferably 50 nm to 1 μm, particularly preferably 100 nm to 5500 nm, wherein the X₅₀ value of the volume-based particle size distribution is measured by laser diffraction.

For this purpose, a sample of the calcium phosphate compounds is first sonicated in an ultrasonic homogenizer with an energy output of 96 W for 9 minutes and then for another 3 minutes in a sample preparation device. The subsequent particle size distribution measurement (laser diffraction) is carried out in a particle size determination instrument at a temperature of 25° C.±0.3° C. and the corresponding values are calculated according to the Mie theory. The measuring instruments used are exclusively commercially available devices.

In a preferred embodiment according to the invention, the calcium phosphate compound is Cas(PO₄)₃(OH) and has a hexagonal crystal lattice in which the length of the a-axis is 0.930 to 0.950 nm, preferably 0.933 to 0.948 nm, particularly preferably 0.936 to 0.945 nm, and the length of the c-axis is 0.680 to 0.700 nm, preferably 0.682 to 0.696 nm, particularly preferably 0.685 to 0.692 nm. The lengths of the a-axis and the c-axis are determined by a Rietveld analysis of the corresponding X-ray powder diffractograms. The X-ray powder diffractograms themselves are obtained by means of a measurement with a conventional powder diffractometer at the routine settings.

In a preferred embodiment of the invention, the at least one calcium phosphate compound is present in aggregated form. Aggregation in this case is understood to be a grouping of molecules or particles to form a larger association, the aggregate. This assembly or aggregate is caused and held together by various forces and/or types of bonding, such as ionic bonding, Van der Waals forces, intermolecular forces or other chemical bonding types. The degree of aggregation and also the size of the aggregate can be determined with the help of scanning electron microscopy.

It has proved advantageous in the context of the invention that the oral care agent contains at least one composition selected from the group comprising antibacterial substances, moisturizing or nourishing substances, desensitizing substances and cleaning agents.

Furthermore, it has been surprisingly shown in the context of the invention that caffeine has a synergistic effect in combination with antibacterial substances, in particular stannous salts, chlorhexidine, cetylpyridinium chloride, triclosan, o-Cymen-5-ol, enzymes, proteins and citrus extract.

It has also been surprisingly shown in the context of the invention that caffeine in combination with moisturizing or nourishing substances, in particular hyaluron, hyaluron salts, allantoin, panthenol, natural extracts (e.g. ectoin) and amino acids (e.g. arginine), has a synergistic effect.

Furthermore, it has been surprisingly shown in the context of the invention that caffeine in combination with desensitizing substances, in particular strontium salts, brings about a synergistic effect.

Similarly, it has been surprisingly shown in the context of the invention that caffeine in combination with cleaning agents, in particular calcium carbonate and sodium bicarbonate, brings about a synergistic effect.

Furthermore, it has been surprisingly shown in the context of the invention that caffeine shows a synergistic effect in combination with soft cleaning agents for plaque removal, in particular calcium carbonate and sodium bicarbonate.

The above-mentioned active substances and groups of active substances may each be combined individually with caffeine, or several of the above-mentioned active substances or groups of active substances may be combined with caffeine.

Furthermore, it has surprisingly been found within the scope of the invention that the oral care agent according to the invention enables a significant activation of the cell metabolism and thus an effective treatment and/or prevention of periodontitis/gingivitis as well as gum recession.

The oral care agent according to the invention is intended for daily use with one to two applications per day.

Insofar as it is present as an oral gel, it does not have to be spat out, but can also be absorbed over a longer period of time of more than 3 minutes, preferably between 5 minutes and 12 hours, particularly preferably between 10 minutes and 1 hour. Due to the gel-like composition, the adhesion of the caffeine directly to the gum or gumline is supported so that the caffeine is in direct contact with it for a longer period of time, thus achieving a high absorption rate.

Here, the caffeine is dosed differently depending on the user group. A daily dose of 0.3 mg caffeine/kg body weight (EFSA NDA Panel (EFSA Panel on Dietetic Products, Nutrition and Allergies, 2015, Scientific opinion on the safety of caffeine, EFSA Journal 2015; 13 (5); 4102, 120 pp. Doi:10.2903/j.efsa.2015.4102) should not be exceeded, which can be absorbed both via a single application and via several applications over the course of a day (wherein 1 to 3 applications per day are preferred).

Preferably, the oral care agent contains caffeine in an amount of from 0.00001 to 4.0% by weight, preferably from 0.0001 to 1.0% by weight and particularly preferably from 0.0005 to 0.001% by weight, in particular 0.005% by weight, each in relation to the total weight of the oral care agent.

Surfactants can be anionic, non-ionic, cationic and zwitterionic surfactants, wherein non-ionic surfactants are often avoided because of their detrimental effects on the antibacterial compound; similarly, cationic and zwitterionic surfactants are often avoided because they stain or darken the teeth. Accordingly, anionic surfactants are used as the cleansing component in the oral care agent, wherein sodium lauryl sulphate and other higher alkyl sulphates having 10 to 18 carbon atoms in the alkyl moiety are commonly used, although other known sulphated and sulphonated surfactants may also be partially or fully used as such.

Preferably, the oral care agent contains at least one surfactant selected from the group consisting of taurates, glycinates, sarcosinates and quaternary ammonium compounds, preferably sodium methylcocoyl taurate, sodium cocoyl glycinate, sodium lauroyl sarcosinate, benzalkonium chloride or cetylpiridinium chloride.

However, since sodium lauryl sulphate is also irritating to the mucous membranes, the oral care agent according to the invention can also be free of sodium lauryl sulphate and instead contain other surfactants that involve less irritation to the mucous membranes than sodium lauryl sulphate, such as taurates, preferably sodium methylcocoyl taurate, glycinates, preferably sodium cocoyl glycinate, sarcosinates, preferably sodium lauroyl sarcosinate or quaternary ammonium compounds, preferably benzalkonium chloride or cetylpyridinium chloride. The specification of the respective surfactants also comprises all variants of the same (different alkyl chains, branches in the alkyl chains, counterions, etc.). Surfactants are important for a toothpaste because they help to distribute the active ingredients in the oral cavity during tooth brushing. Surfactants are also important for consumer convenience.

Sodium lauryl sulphate-free oral care agents may be important, for example, for people with sensitive or irritated gums (children, periodontitis patients, people with xerostomia/hyposalivation). To combat plaque, sodium lauryl sulphate and silica are the most potent active ingredients in cosmetic oral care products. In the present invention, the disadvantages of these two active ingredients (sodium lauryl sulphate: irritating to the mucous membrane; silica: potentially damaging to teeth, not remineralizing) could be overcome for the first time; i.e. a cleaning that is equally gentle on the mucous membrane and tooth, but effective, with simultaneous effective remineralizing, antibacterial and caries-protecting action.

In particular, it was unexpectedly found that the oral care agent according to the invention advantageously mineralizes smaller caries lesions and/or repairs microfine defects in tooth enamel, in particular also in patients with dry mouth/saliva deficiency. Furthermore, a protective layer can be applied to the tooth by means of the composition according to the invention and, furthermore, it was found that the enamel exhibits advantageous solubility and resistance to acid after the application of the composition according to the invention. In addition, the present composition can dispense with antimicrobial substances (such as chlorhexidine) as ingredients. In this way, excellent caries prevention can be ensured without, for example, disturbing/disrupting the bacterial balance in the oral cavity and without risking unpleasant side effects.

In particular, it was unexpectedly found that the oral care agent according to the invention prevents caries and can also mineralize lesions down to deeper layers of the tooth, in particular the enamel. Furthermore, the oral care agent according to the invention can be used to apply a protective layer on the tooth and over exposed dentin and, in particular, to close open dentinal tubules. Furthermore, it was found that the enamel showed significantly reduced or undetectable structural damage after application. In addition, fluoride application can be completely dispensed with in the present oral care agent; the present oral care agent is free of fluorides. In this way, the above positive aspects can be ensured without, for example, disturbing/disrupting the bacterial balance in the oral cavity and without risking undesirable side effects, which can occur, for example, when using oral care products containing fluoride. The subject matter of the present invention is an oral care agent for mineralizing teeth, in particular tooth enamel, to a depth of 200 μm, preferably to 100 μm (deep mineralization).

The oral care agent according to the invention comprises calcium phosphate compounds in an amount from 0.01 to 80% by weight, preferably 0.1 to 20% by weight, particularly preferably 1 to 10% by weight and in particular 1 to 5% by weight, in relation to the total weight of the oral care agent. For mouth rinses, the preferred amount of calcium phosphate compounds is 1 to 5% by weight, for toothpastes it is 10 to 20% by weight, in each case based on the total weight.

The oral care agent according to the invention preferably contains at least one calcium compound, selected from the group consisting of calcium carbonate, calcium chloride, calcium bromide, calcium nitrate, calcium acetate, calcium gluconate, calcium lactate, calcium tartrate and hydrates and mixtures thereof, preferably calcium carbonate.

In a preferred embodiment of the invention, the oral care agent contains a tin salt and/or chlorhexidine and/or triclosan and/or an amino acid and/or an enzyme and/or hyaluron and/or a strontium salt and/or sodium hydrogen carbonate.

The tin salt can be tin chloride, for example. Arginine, for example, is an amino acid. Enzymes include lactoferrin or lysozyme, for example.

Alternatively, it is also possible that the oral care agent is free of tin salts and/or chlorhexidine and/or cetylpyridinium chloride and/or triclosan.

The oral care agent according to the invention preferably does not contain perlite. Perlite is a cleaning agent with high relative hardness and can damage the teeth.

The oral care agent according to the invention preferably does not contain aluminum compounds. Aluminum compounds can be potentially toxic.

The oral care agent according to the invention preferably does not contain diamond particles. Diamond is a cleaning agent with high relative hardness and can damage the teeth.

The oral care agent according to the invention preferably does not contain any cellulose fibres (microcrystalline cellulose=cleaning agent). Cellulose fibres often lead to an unpleasant mouthfeel, severely limiting user compliance.

The oral care agent according to the invention preferably does not contain sodium hydrogen carbonate. Sodium hydrogen carbonate has only a very low cleaning performance.

The oral care agent according to the invention preferably does not contain glucosides. Glucosides can break down glucose, which promotes the undesirable development of caries.

The oral care agent according to the invention does not contain abrasive polymer particles/“microbeads” (e.g. polyethylene beads). Such polymer particles can have harmful effects on the environment.

The oral care agent according to the invention preferably does not contain any sulphates, such as sodium lauryl sulphate, or sulphonates.

Furthermore, the oral care agent according to the invention preferably does not contain tin salts, such as stannous fluoride and stannous chloride, as these can lead to discoloration of the teeth.

The oral care agent according to the invention also preferably does not contain peroxides, as these are irritating to the mucous membranes.

Furthermore, the oral care agent according to the invention preferably also contains no enzymes and is therefore a vegan product.

The oral care agent according to the invention is used for mineralization of teeth up to a depth of 200 μm, preferably up to 150 μm, in particular up to 100 μm. Such mineralization of teeth down to these depths is called deep mineralization, since in this case not only the tooth surface areas down to a depth of approximately 30 μm, as described in the prior art, but also deeper lying areas of the tooth are mineralized.

It was found that the oral care agent according to the invention can be used in the treatment and/or prevention of numerous dental diseases.

In a preferred embodiment of the invention, the oral care agent according to the invention can be used for the treatment of (dental) diseases/conditions selected from caries, dental erosion, dental abrasion, attrition, bruxism, molar incisor hypomineralization (MIH), amelogenesis imperfecta, dentinogenesis imperfecta and fluorosis.

The term caries is familiar to the person skilled in the art. Thus, caries is generally understood to be a destructive disease of the hard tissues of the teeth, enamel and dentin.

Dental erosion refers to damage to the tooth structure caused by acids, in other words defects in the enamel and/or dentin caused by dental erosion, which, if treated too late, can lead to irreversible damage.

(Dental) abrasion is the loss of hard tooth tissue through friction. Attrition is a subform of abrasion, namely the loss of tooth structure by reflexive contact with the teeth.

Bruxism is the unconscious grinding or clenching of the teeth, usually at night but also during the day, which can result in wear and tear not only of the teeth but also of the periodontium and chewing muscles.

Molar incisor hypomineralization (MIH), which is also known as “chalky teeth”, is an enamel disorder, i.e. structural damage to the enamel.

Amelogenesis imperfecta is considered to be a genetic disorder in which there is a disturbance in the formation of tooth enamel. As a result, the teeth have an increased risk of caries formation and are particularly sensitive to temperature.

Dentinogenesis imperfecta is an autosomal dominant inherited maldevelopment/structural disorder of the tooth dentition that occurs in approximately 1 in 8000 people and results in severe abrasion of the teeth.

Tooth fluorosis (also dental fluorosis) is a non-inflammatory disease (“speckled teeth”) caused by excessive fluoride intake, in particular during the ontogenetic development of the teeth. It was found that the use according to the invention can prevent the above-mentioned (dental) and/or at least significantly slow their progression and/or achieve complete restoration of the tooth substance, in particular the hard tooth substance. In particular, it was found that the enamel shows significantly reduced or no longer detectable structural damage due to the use according to the invention or after its application.

In a preferred embodiment, the oral care agent according to the invention can be used to treat a Code 3 or Code 4 caries, preferably a Code 3 caries, determined according to the International Caries Detection and Assessment System (ICDAS).

According to the International Caries Detection and Assessment System (ICDAS), caries is classified into different codes (levels), wherein the higher the code, the more severe the decay on the tooth and consequently its impact on that tooth.

In the case of a Code 0 caries, no signs of caries are visible after drying in the air for about 5 seconds.

In the case of a Code 1 caries, the first visual changes in the enamel surface are visible after the tooth has dried. The changes can be opacities and whitish or brownish discoloration.

In the case of a Code 2 caries, clear visual changes in the enamel surface are already present on the moist tooth. These changes can be opacities in the sense of a white spot lesion and/or brownish carious discoloration in the fissures/grooves and must still be visible on the dried tooth.

In the case of a Code 3 caries, demineralization or loss of the enamel structure is present without visible changes to the dentin. The opacities and/or brownish or black carious changes extend beyond the border of the fissures/grooves and are visible even after the tooth has dried. If necessary, a WHO probe can be carefully passed over the enamel defect to palpate the discontinuity of the enamel surface.

In the case of a Code 4 caries, there is shading in the dentin, with or without enamel collapse. The shading can be greyish, bluish or brownish.

In the case of a Code 5 caries, clear cavity formation with visible dentin can be seen.

The enamel loss is clearly visible on the dried tooth. If necessary, the WHO probe can be used to feel the exposed dentin.

In the case of a Code 6 caries, there is extensive cavity formation, wherein the dentin is clearly visible in the width and depth of the tooth. At least half of the enamel surface is cariously destroyed. The pulp may be affected.

In a preferred embodiment, the oral care agent according to the invention can be used for both cosmetic and medical purposes. This means that it can be used, for example, not only for the treatment of the above-mentioned (dental) diseases, but also for cosmetic purposes such as enhancing the appearance of teeth.

In a preferred embodiment, the oral care agent according to the invention is used on persons of all ages, including children, in the case of children preferably on children aged from 6 months to 14 years, in particular on children aged from 10 months to 12 years.

In a preferred embodiment, the Cas(PO₄)₃(OH) contained in the oral care agent according to the invention is the only apatite component of the oral care composition.

In a preferred embodiment, the oral care agent according to the invention comprises 0.01 to 80% by weight, preferably 0.2 to 40% by weight, more preferably 0.5 to 30% by weight, in particular 1.0 to 20% by weight calcium compound. In a preferred embodiment of the invention, the oral care agent according to the invention can comprise 1% by weight, 2% by weight, 3% by weight, 4% by weight, 5% by weight, 6% by weight, 7% by weight, 8% by weight, 9% by weight, 10% by weight, 11% by weight, 12% by weight, 13% by weight, 14% by weight, 15% by weight, 20% by weight or 25% by weight calcium phosphate compounds.

In a preferred embodiment, the oral care agent according to the invention comprises one or more calcium salts, which have a solubility of at least 10 mg/l H₂O at 20° C. The solubility is determined according to methods known to the person skilled in the art or can be obtained from the relevant technical literature.

In a preferred embodiment, the oral care agent according to the invention can contain one or more pharmaceutical or cosmetic ingredients. These pharmaceutical or cosmetic ingredients are described, for example in Toothpastes, Monographs in Oral Science, Vol. 23, 1st edition, 2013.

Preferably, the one or more pharmaceutical or cosmetic ingredients comprise xylitol, antimicrobial substances, pH regulators, abrasives, flavoring agents and moisturizing agent, in particular xylitol, pH regulators, abrasives and flavoring agents.

Xylitol can minimize the number of caries bacteria and inhibit their growth. Furthermore, xylitol can stimulate the flow of saliva. The increased amount of saliva results in an increased amount of phosphate. This phosphate can react together with the calcium (ions) from the oral care agent according to the invention to form hydroxyapatite. The oral care agent according to the invention can contain xylitol in an amount of from 0.5 to 15% by weight, preferably from 1 to 10% by weight, in particular approximately 7.0% by weight in relation to the total weight of the oral care agent. In addition to xylitol, the oral care agent according to the invention can contain other sugar alcohols such as sorbitol.

Antimicrobial substances are substances that can kill microorganisms, such as bacteria, or greatly reduce their proliferation. In addition to antimicrobial substances with a non-specific defense against bacteria and fungi, there are also substances that are only effective against specific bacteria, for example. The use of antimicrobial substances can also combat bad breath, for example. Preferably, antimicrobial substances may be present in an amount of from 0.01 to 2.0% by weight, preferably from 0.05 to 1.0% by weight, in the oral care agent according to the invention. Examples of antimicrobial substances used in oral care are zinc compounds such as zinc chloride and zinc citrate, as well as cetylpyridinium chloride, essential oils and surfactants.

In a particularly preferred embodiment, the oral care agent according to the invention is free of fluorides and/or tin salts and/or chlorhexidine and/or cetylpyridinium chloride and/or triclosan.

pH regulators are substances that can adjust a certain pH value range, preferably a range from pH 5.5 to 8.0. If the composition is too acidic, there would be a risk of demineralization of the tooth structure (erosion).

Examples of pH regulators include acetic acid, acetates, lactic acid, lactates, malic acid, malates, fumaric acid, citric acid, citrates, tartaric acid, tartrates, orthophosphates, di-, tri- and polyphosphates, hydrochloric acid, chlorides, sulphuric acid, sulphates, hydroxides, oxides, adipic acid, adipates, gluconic acid, gluconates, phosphoric acid, calcium carbonate or a hydrate thereof. A preferred example of a pH regulator that can be added when a lower pH is desired is phosphoric acid (H₃PO₄).

In a further preferred embodiment, the oral care agent according to the invention contains a pH regulator in an amount of from 0.05 to 3.0% by weight, more preferably from 0.1 to 2.5% by weight, particularly preferably 0.2 to 1.5% by weight, each in relation to the total weight of the oral care agent.

In a preferred embodiment, the oral care agent according to the invention contains one or more flavoring agent, which can give it the desired taste. This one or these several flavoring agents may be natural, nature-identical, synthetic flavoring agents and/or mixtures thereof. Examples of flavoring agents are limonene, geraniol, citronellol and eugenol. In addition, flavoring agents can stimulate saliva, wherein the moisture of the saliva can have a positive influence on the remineralization of the tooth. An example of a saliva-stimulating flavoring agent is pellitorin, in particular trans-pellitorin.

The oral care agent according to the invention can preferably contain flavoring agents in an amount of 0 to 5% by weight, preferably 0.1 to 3% by weight in relation to the total weight of the oral care agent according to the invention.

Moisturizing agents are additives that prevent the oral care agent according to the invention from drying out by binding water added during manufacture (i.e. preventing evaporation) or by attracting moisture from the air during storage. Examples of moisturizing agents are glycerin, propane-1,2-diol, hexane-1,2-diol, egg yolk, aloe vera gel, honey, molasses, in particular glycerin and hexane-1,2-diol. The oral care agent according to the invention can preferably contain moisturizing agents in an amount of 0 to 25% by weight, preferably 0.1 to 20% by weight in relation to the total weight of the oral care agent according to the invention.

In a preferred embodiment, the oral care agent according to the invention contains
0.01 to 80% by weight, preferably 0.2 to 40% by weight, more preferably 0.5 to 30% by weight, in particular 1.01 to 20% by weight calcium phosphate compounds, preferably hydroxyapatite
0.00001 to 4.0% by weight, preferably 0.0001 to 1.0% by weight, particularly preferably 0.0005 to 0.1% by weight caffeine,
0.3 to 3% by weight surfactants, preferably approximately 1.0% by weight sodium cocoyl glycinate or sodium methyl cocoyl taurate,
0.5 to 15% by weight, preferably 1 to 10% by weight, in particular 7.0% by weight xylitol,
0 to 2.0% by weight, preferably 0.01 to 2.0% by weight, more preferably 0.05 to 1.0% by weight antimicrobial substance, in particular cetylpyridinium chloride or zinc chloride,
0 to 5% by weight, preferably 0.3 to 2.0% by weight pH regulator, in particular phosphoric acid,
0 to 5% by weight, preferably 0.1 to 3% by weight flavoring agent,
from 0 to 25% by weight, preferably 0.1 to 20% by weight moisturizing agent, in particular glycerin and/or hexane-1,2-diol,
   wherein the percentages by weight relate to the total weight of the oral care agent. The rest is distilled water, if necessary.

When the oral care agent is in the form of an oral gel, it contains gelling agents, wherein the gelling agent consists of synthetic and/or natural polymeric materials.

Here, it is advantageous that the gelling agent is selected from the group containing cellulose and cellulose derivatives such as methyl cellulose, ethyl cellulose, hydroxyethyl cellulose, hydroxypropyl cellulose, hydroxypropyl methyl cellulose, carboxymethyl cellulose, sodium carboxymethyl cellulose, agaraose, agar, pectins, sclerotium gum, xanthan gum, guar gum, carrageenan, alginic acid, polyvinyl alcohol, polyvinyl pyrrolidone and mixtures thereof.

Preferably, such an oral gel contains gelling agent in an amount of from 1.0 to 4.0% by weight, particularly preferably from 1.5 to 3.0% by weight and in particular approximately 2.0% by weight, each in relation to the total weight of the oral gel.

The pH value is in a range from pH 5.0-9.0, preferably 6.5 to 7.5.

### BRIEF DESCRIPTION OF THE DRAWINGS

FIG. 1 shows the results of P13-A Experiment 1;

FIG. 2 shows the results of P13-A Experiment 2;

FIG. 3 shows the results of P13-B Experiment 1;

FIG. 4 shows the results of P13-B Experiment 2;

FIG. 5 shows the results of CyQuant Experiments 1&2 Caffeine;

FIG. 6 shows the results of Results of Experiment 1-Q-PCR-VEGF-24h;

FIG. 7 Results of Experiment 2-Q-PCR-VEGF-24h;

FIG. 8 Results of Experiments 1&2-ELISA-VEGF-48h;

FIG. 9 Results of Experiment 1-Q-PCR-TGFβ1-24h;

FIG. 10 Results of Experiment 2-Q-PCR-TGFβ1-24h;

FIG. 11 Results of Experiment 1-Q-PCR-TGFβ2-24h;

FIG. 12 Results of Experiment 2-Q-PCR-TGFβ2-24h;

FIG. 13 Results of Experiment 1-Q-PCR-GM-CSF-24h;

FIG. 14 Results of Experiment 2-Q-PCR-GM-CSF-24h; and

FIG. 15 Results of Experiments 1&2-ELISA-GM-CSF-48h.

### DETAILED DESCRIPTION OF PREFERRED EMBODIMENTS

In the following, the invention is explained by means of examples.

Exemplary embodiments for the composition of the oral care agent according to the invention in the form of toothpastes (in % by weight):

| | Exemplary | Exemplary | Exemplary | Exemplary |
|---|---|---|---|---|
| | embodi- | embodi- | embodi- | embodi- |
| | ment 1 | ment 2 | ment 3 | ment 4 |
| Glycerine | 10.0 | 5.0 | 10.0 | 14.0 |
| Sorbitol | 4.0 | 3.0 | 10.0 | 6.0 |
| Hydroxyapatite | 30.0 | 20.0 | 10.0 | 5.0 |
| Caffeine | 0.001 | 0.0005 | | 0.005 |
| Theobromine | | 0.0005 | | |
| Theophylline | | | 0.001 | |
| Xylitol | | 2.0 | 8.0 | 10.0 |
| Cellulose gum | 7.0 | 1.0 | 1.0 | 3.0 |
| Hydroxyethyl | | 0.2 | 1.0 | 0.5 |
| cellulose | | | | |
| Carboxymethyl | 0.1 | 0.2 | 1.0 | 0.5 |
| cellulose | | | | |
| Sodium methyl | 1.0 | 2.0 | 0.5 | 2.0 |
| cocoyl taurate | | | | |
| Sodium | | | 0.5 | |
| sulphate | | | | |
| 1,2-Hexanediol | | | 0.2 | |
| Caprylyl glycol | | | 0.5 | |
| Flavoring | 0.5 | | 0.3 | 1.5 |
| Sodium cocoyl | | | 0.2 | |
| glycinate | | | | |
| Sodium chloride | | 0.7 | | 0.3 |
| Benzyl alcohol | | 0.5 | | |
| Phenoxyethanol | 0.3 | 0.4 | 0.1 | 0.3 |
| Potassium sorbate | 0.2 | | | |
| Sodium benzoate | | 1.0 | | 1.0 |
| Allantoin | | | 0.5 | 1.0 |
| Sodium lauryl | | 0.1 | 0.2 | 0.2 |
| sulphate | | | | |
| Sodium myristoyl | 1.0 | 1.5 | | 2.0 |
| sarcosinate | | | | |
| Sodium saccharin | 0.5 | 0.5 | 0.5 | 0.5 |
| Tocopheryl | | 0.1 | | 1.0 |
| acetate | | | | |
| Zinc chloride | 0.1 | 0.1 | | 0.5 |
| Carrageenan | 0.1 | 3.0 | | 0.5 |
| Xanthan gum | | 0.2 | 0.3 | 0.3 |
| Remaining: | | | | |
| demineralized | | | | |
| water | | | | |

### Study 1: Effect of Caffeine on the Biological Functions of Human Epidermal HaCaT Keratinocytes (Viability Study)

To investigate the effect of caffeine on the biological functions of human epidermal HaCaT keratinocytes, cell experiments were performed on human epidermal HaCaT keratinocytes, wherein the following studies were carried out:
1. P13-A biological assay 1 with two independent studies
2. P13-B biological assay 2 with two independent studies.

The initial cell count was 5000 cells/well in 100 μl culture medium.

The culture medium was HaCaT medium (DMEM with the addition of 10% FBS, 1% penicillin-streptomycin, 0.5% fungizone).

The treatment groups were as follows:
1. Control group (CTRL)
2. 0.00005% caffeine
3. 0.0001% caffeine
4. 0.0005% caffeine
5. 0.001% caffeine
6. 0.005% caffeine

The trial durations were 24, 48 and 72 hours.

The study took the form of a colorimetric MTT study.

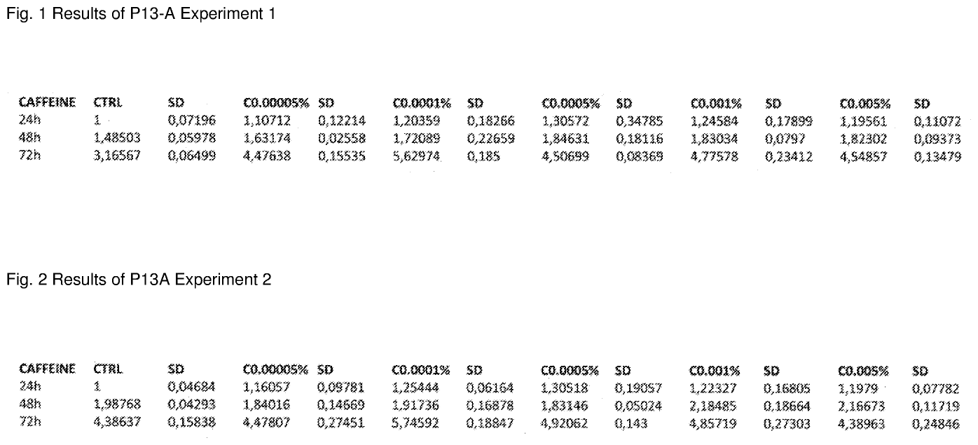

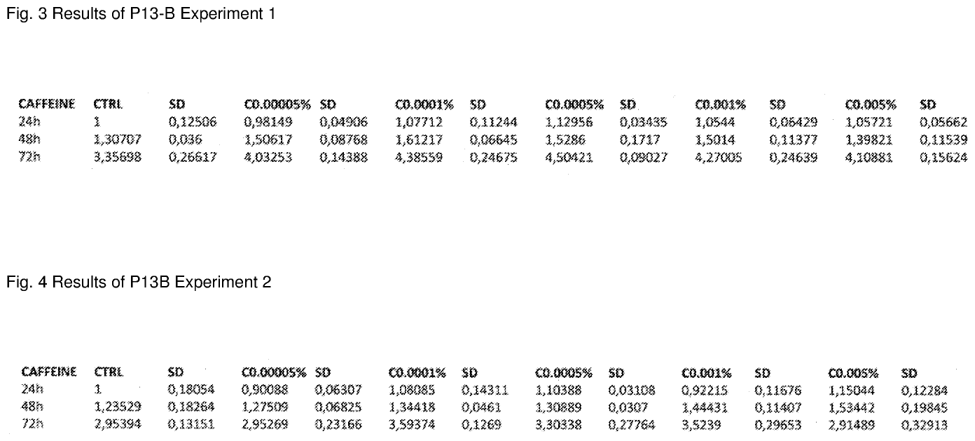

The results are presented in
FIG. 1 Results of P13-A Experiment 1
FIG. 2 Results of P13-A Experiment 2
FIG. 3 Results of P13-B Experiment 1
FIG. 4 Results of P13-B Experiment 2.

The study found that none of the concentrations tested showed cytotoxicity even after 72 hours of testing and that certain concentrations of caffeine increased the number of viable cells.

### Study 2: Effect of Caffeine on the Biological Functions of Human Epidermal HaCaT Keratinocytes (Cell Proliferation Study)

To investigate the effect of caffeine on cell proliferation of human epidermal HaCaT keratinocytes, human HaCaT keratinocytes were subjected to a CyQUANT viability assay, wherein the following series of experiments were performed:
1. P22 Experiment 1 (biological assay 1)
2. P24 Experiment 2 (biological assay 2).

The initial cell count was 5000 cells/well in 1,500 μl culture medium.

The culture medium was HaCaT medium (DMEM with the addition of 10% FBS, 1% penicillin-streptomycin, 0.5% fungizone).

The treatment groups were as follows:
1. Control group (CTRL)
2. 0.00005% caffeine
3. 0.0005% caffeine
4. 0.005% caffeine

The trial durations were 24, 48 or 72 hours.

The study was carried out by fluorometric CyQUANT study, wherein the DNA content was measured, reflecting cell proliferation.

The results are presented in

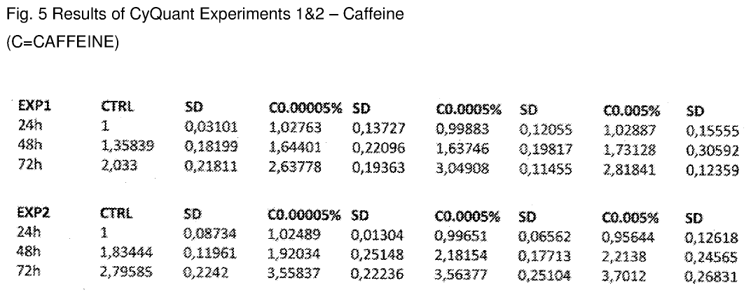

FIG. 5 Results of CyQuant Experiments 1&2 Caffeine

The study shows that caffeine appears to promote cell proliferation; the best proliferation-enhancing effects were found after 72 hours.

### Study 3: Effect of Caffeine on the Biological Functions of Human Epidermal HaCaT Keratinocytes (Cell Proliferation Study)

To investigate the effect of caffeine on the biological functions of human epidermal HaCaT keratinocytes, human HaCaT keratinocytes were subjected to Q-PCR and ELISA, wherein the following series of experiments were performed:
1. For Q-PCR: Determination of VEGF, HGF and EGF mRNA expression in cell lysates
2. For ELISA: Determination of VEGF, HGF and EGF protein synthesis/secretion into the culture medium

The initial cell count was
1. for Q-PCR: 140,000 cells/well in 1500 μl culture medium
2. for ELISA: 140,000 cells/well in 1.500 μl culture medium.

The culture medium was HaCaT medium (DMEM with the addition of 10% FBS, 1% penicillin-streptomycin, 0.5% fungizone).

The treatment groups were as follows:
1. Control group (CTRL)
2. 0.00005% caffeine
3. 0.0005% caffeine
4. 0.005% caffeine

The trial duration for
1. Q-PCR was 24 hours (single treatment at time 0 h)
2. ELISA was 48 hours (double treatment at time 0 h and 24 h without changing the medium).

The study was carried out
1. for Q-PCR by determining VEGF mRNA expression in cell lysates
2. for ELISA by determining VEGF protein synthesis/secretion in the culture medium.

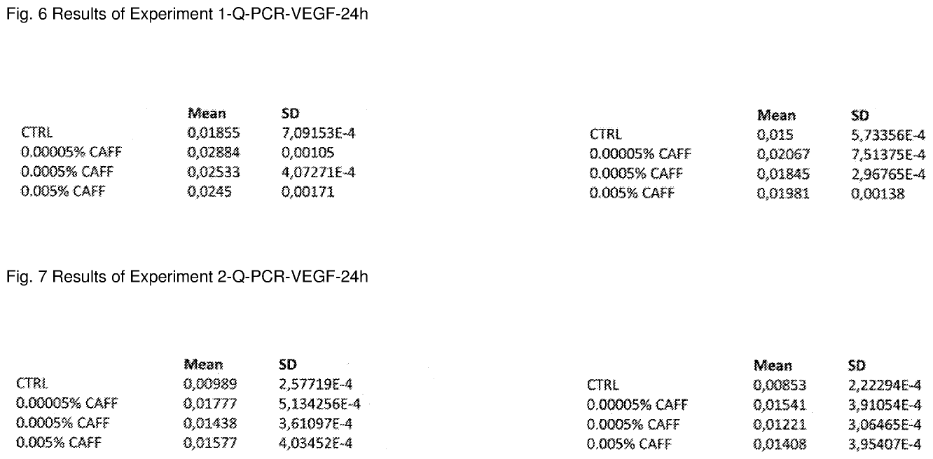

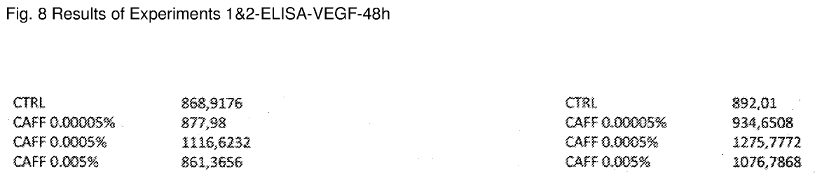

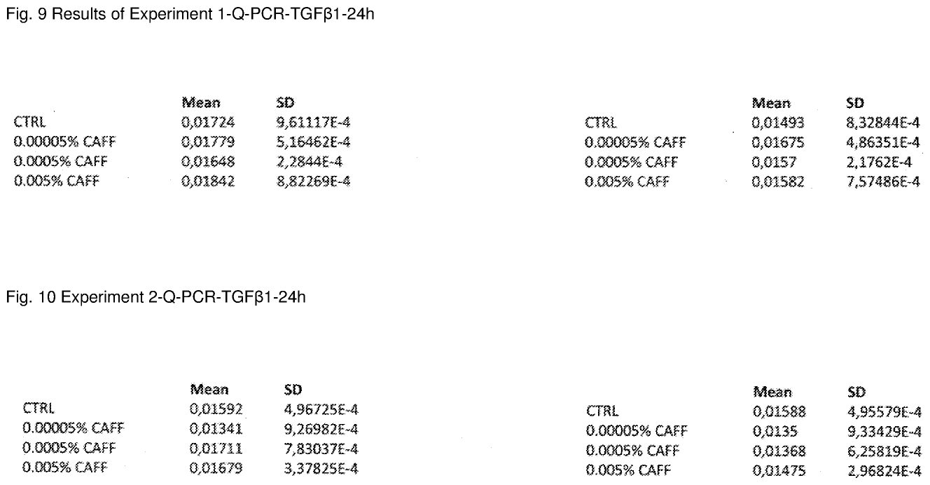

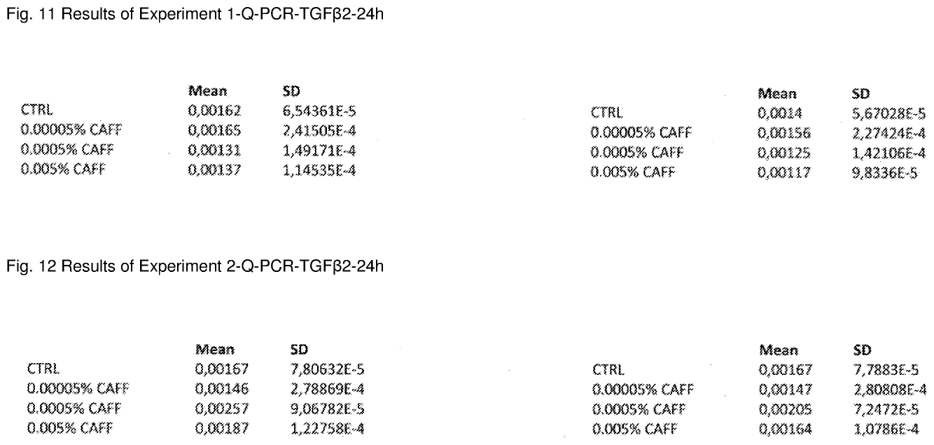

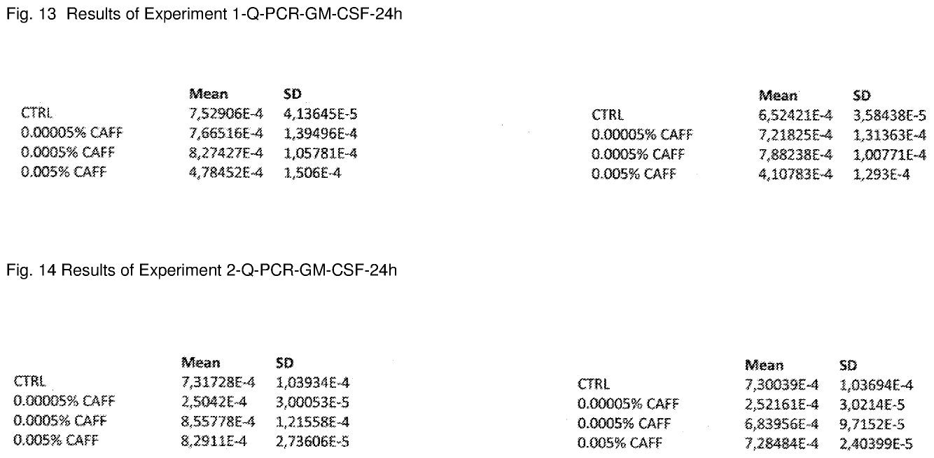

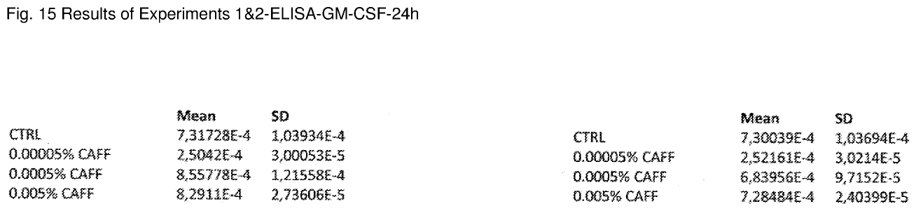

The results are presented in
FIG. 6 Results of Experiment 1-Q-PCR-VEGF-24h
FIG. 7 Results of Experiment 2-Q-PCR-VEGF-24h
FIG. 8 Results of Experiments 1&2-ELISA-VEGF-48h
FIG. 9 Results of Experiment 1-Q-PCR-TGFβ1-24h
FIG. 10 Results of Experiment 2-Q-PCR-TGFβ1-24h
FIG. 11 Results of Experiment 1-Q-PCR-TGFβ2-24h
FIG. 12 Results of Experiment 2-Q-PCR-TGFβ2-24h
FIG. 13 Results of Experiment 1-Q-PCR-GM-CSF-24h
FIG. 14 Results of Experiment 2-Q-PCR-GM-CSF-24h
FIG. 15 Results of Experiments 1&2-ELISA-GM-CSF-48h.

The study found the following:
Q-PCR (24 hours treatment time)—Experiments 1 and 2: All caffeine concentrations appear to increase mRNA expression of VEGF compared to the control group.
ELISA (48 hours treatment time)—Experiments 1 and 2: 0.0005% caffeine increases VEGF protein production/secretion compared to the control group in both experiments. In Experiment 2, 0.005% caffeine also showed an effect.

Taken together, this data suggests that certain concentrations of caffeine increase the synthesis and production/secretion of VEGF in cultured human epidermal HaCaT keratinocytes.

The expression of HGF-specific mRNA, HGF-specific protein, TNFα-specific mRNA, TNFα-specific protein and TGFβ1-specific protein was below the detection limit in both experiments.

The experiments regarding GM-CSF show the following:
Q-PCR-Experiment (24 h treatment time)—Experiments 1 and 2: All tested caffeine concentrations apparently increased the mRNA expression of VEGF compared to the control group.
ELISA (48 h treatment time)—Experiments 1 and 2: 0.0005% caffeine increased VEGF protein production/secretion compared to the control group in both experiments. In Experiment 2 0j.005% caffeine also showed an effect.

Taken together, this data strongly suggests that certain concentrations of caffeine increase the synthesis/production/secretion of VEGF in cultured human epidermal HaCaT keratinocytes.
Q-PCR-Experiment (24 h treatment time)—Experiments 1 and 2: None of the tested caffeine concentrations changed the mRNA expression of TGFβ1. However, in Experiment 2, certain caffeine concentrations apparently increase mRNA expression of TGFβ2.
ELISA (48 h treatment time)—Experiments 1 and 2: The level of TGFβ1-specific protein was below the detection limit in both experiments.

Taken together, this data suggests—despite the contradictory results in the two Q-PCR experiments—that caffeine most likely does not alter the synthesis/production/secretion of TGFβ2 in cultured human epidermal HaCaT keratinocytes.

The experiments regarding TGFβ1 showed the following:
Q-PCR-Experiment (24 h treatment time)—Experiments 1 and 2: None of the tested caffeine concentrations changed the mRNA expression of TGFβ1.
ELISA (48 h treatment time)—Experiments 1 and 2: The level of TGFβ1-specific protein was below the detection limit in both experiments.

Taken together, this data suggests that caffeine does not alter the synthesis/production/secretion of TGFβ1 in cultured human epidermal HaCaT keratinocytes.

1. An oral care agent, in particular toothpaste or mouthwash, wherein the oral care agent contains
caffeine and
a calcium phosphate compound, preferably in particulate form, selected from the group consisting of monocalcium phosphate monohydrate (MCPM), anhydrous monocalcium phosphate (AMCP), octacalcium phosphate (OCP), amorphous calcium phosphate (ACP), calcium-deficient hydroxyapatite (CDHA), hydroxyapatite (HA or HAP) and tetracalcium phosphate (TTCP), particularly preferably hydroxyapatite.

2. An oral care agent, wherein the oral care agent contains at least one composition selected from the group comprising antibacterial substances, moisturizing or nourishing substances, desensitizing substances and cleaning agents.

3. The oral care agent according to claim 1, wherein the oral care agent contains caffeine in an amount of from 0.00001 to 4.0% by weight, preferably from 0.0001 to 1.0% by weight and particularly preferably from 0.0005 to 0.1% by weight,
each in relation to the total weight of the oral care agent.

4. The oral care agent according to claim 1, wherein the antibacterial substances comprise tin salts, chlorhexidine, cetylpyridinium chloride, triclosan, o-Cymen-5-ol, enzymes, proteins and citrus extract.

5. The oral care agent according to claim 1, wherein the moisturizing or nourishing substances comprise hyaluron, hyaluron salts, allantoin, panthenol, natural extracts and amino acids.

6. The oral care agent according to claim 1, wherein the desensitizing substances comprise strontium salts.

7. The oral care agent according to claim 1, wherein the cleaning agents comprise calcium carbonate and sodium carbonate.

8. The oral care agent according to claim 1, wherein the oral care agent contains at least one surfactant selected from the group consisting of taurates, glycinates, sarcosinates and quaternary ammonium compounds, preferably sodium methylcocoyl taurate, sodium cocoyl glycinate, sodium lauroyl sarcosinate, benzalkonium chloride or cetylpiridinium chloride.

9. The oral care agent according to claim 1, wherein the oral care agent comprises calcium phosphate compounds in an amount from 0.01 to 80% by weight, preferably 0.1 to 20% by weight, particularly preferably 1 to 10% by weight and in particular 1 to 5% by weight, in relation to the total weight of the oral care agent.

10. The oral care agent according to claim 1, wherein the oral care agent contains at least one calcium compound, selected from the group consisting of calcium carbonate, calcium chloride, calcium bromide, calcium nitrate, calcium acetate, calcium gluconate, calcium lactate, calcium tartrate and hydrates and mixtures thereof, preferably calcium carbonate.

11. The oral care agent according to claim 1, wherein the oral care agent is free of fluorides and/or tin salts and/or chlorhexidine and/or cetylpyridinium chloride and/or triclosan.
